(19) United States
(12) Patent Application Publication
(10) Pub. No.: US 2018/0360712 A1
(43) Pub. Date: Dec. 20, 2018

(54) COSMETIC COMPOSITION AND USE THEREOF

(71) Applicant: UNDERSKIN FARMACEUTICAL LTDA, Hortolandia (BR)

(72) Inventors: Fernanda Gobbo CHAVES, Hortolandia (BR); Luiz Felipe De Oliveira STEHLING, Hortolandia (BR); Silvana MASIERO, Hortolandia (BR); Juliana Maria BARBOSA, Hortolandia (BR); Camila Pereira SANTOS, Hortolandia (BR); Leticia Khater COVESI, Hortolandia (BR); Matheus PAVANI, Hortolandia (BR)

(21) Appl. No.: 16/062,551

(22) PCT Filed: Dec. 15, 2015
(86) PCT No.: PCT/BR2015/050256

§ 371 Date: Jun. 14, 2018

(51) Int. Cl. A61K 8/365; A61K 8/362; A61K 8/368; A61K 8/73; A61K 8/60; A61K 8/34; A61Q 19/08

(52) U.S. Cl. CPC A61K 8/365; A61K 8/362; A61K 8/368; A61K 2800/5922; A61K 8/602; A61K 8/34; A61Q 19/08; A61K 8/73

(57) ABSTRACT

It is described here a cosmetic composition and its use for the reduction of dermatological disorders resulting from the use of alpha-hydroxy acids (AHAs), while maintaining their effectiveness in the cell renewal process. In particular, said composition comprises one or more AHA compounds as active ingredients, wherein preferably the AHA compounds are glycolic acid, lactic acid, mandelic acid and malic acid. Additionally, said composition comprises one or more polyhydroxy acid (PHAs) compounds, wherein preferably the PHA compound is lactobionic acid.

### FIELD OF THE INVENTION

The present invention relates to a cosmetic composition containing alpha-hydroxyacids (AHAs) and their use. Said composition has a soothing action and performs cell renewal function, without exhibiting the adverse effects related to skin irritation. In particular, the formulation plays the role of histamine synthesis prevention/inhibition by combining polyhydroxy acid with alpha-hydroxy compounds, preventing irritation, pruritus and erythema reactions. Specifically, the alpha-hydroxy compounds comprise glycolic acid, lactic acid, malic acid and mandelic acid and the polyhydroxy acid comprises lactobionic acid.

### BACKGROUND OF THE INVENTION

Since the skin is the largest organ in the human body, it is continually exposed to a variety of agents, which can result in changes at the molecular level, that is, sagging and wrinkling. Such agents may be caused by the external environment, including solar radiation and air pollutants, and by the internal environment, including reactive compounds originating during the normal metabolism of the organism or as a result of an external disturbance.

Cell renewal is vital for skin health as it is responsible for reducing the thickening of the corneal layer, leaving the skin smoother and more uniform. Over the years, this process of cellular renewal becomes less efficient, resulting in loss of skin elasticity and consequent skin aging. For this reason, cosmetic formulations with antiaging properties have been extensively studied and developed recently.

Compositions containing alpha-hydroxy acids (AHAs) have been shown to be quite effective against skin aging. These compounds are used in dermatological treatments, where their topical application stimulates the cellular renewal process, guaranteeing the revitalization of dim, devitalized and photoaged skins.

However, there are still difficulties in the treatment of dermatological disorders, because although they present cosmetic benefits, such as improvement in the treatment of age-related marks, wrinkles and other signs of aging, AHAs may present adverse effects related to skin irritation when used in high concentrations (up to 10% by weight of a cosmetic formulation intended for domestic use).

Nardin & Guterres (1999) show that products containing AHAs are widely used for skin anti-aging treatment. Furthermore, even if a pH adjustment of the formulation is performed to make the treatment compatible with the skin pH (4.2-5.6), many AHA-containing products are irritating to sensitive or atopic skin. The main adverse reactions caused by AHAs include severe erythema, swelling, burning, itching, among others.

Document WO0015179 discloses the use of compositions containing alpha-hydroxy acid and, in addition, petroselinic acid.

The polyhydroxy acids (PHAs) are alpha-hydroxy acids that, in addition to cell renewal, promote a humectant and antioxidant action.

The main polyhydroxy acid compounds are lactobionic acid, gluconic acid and gluconolactone.

Lactic acid can be found in milk. This compound has strong antioxidant activity and is widely used in solutions for the preservation of transplanted organs.

Although the use of alpha-hydroxy acid-containing compositions in dermatological treatments that promote cell renewal is known from the prior art, it is imperative to develop a cosmetic formulation that avoids the appearance of skin irritation reactions, while maintaining efficacy in treatment.

### SUMMARY OF THE INVENTION

The present invention aims to provide a cosmetic formulation containing a combination of polyhydroxy acid (PHA) with alpha-hydroxy acids (AHAs) and their use for the reduction of dermatological disorders resulting from the use of AHAs, while maintaining their effectiveness in the cell renewal process.

A first embodiment of the present invention relates to a cosmetic composition containing alpha-hydroxyacids (AHAs) with soothing action. The novel composition disclosed herein is formulated with one or more AHA compounds and one or more polyhydroxy acidic compounds.

Most preferably, the composition of the present invention is formulated with at least four AHA compounds and at least one polyhydroxy acid compound.

In a preferred embodiment, the alpha-hydroxy acids used in the formulation of the composition are glycolic acid, lactic acid, mandelic acid and malic acid, these compounds being present in a concentration of 0.1 to 10.0% based on the final weight of the composition. Furthermore, the polyhydroxy acid is lactobionic acid, as its concentration ranges from 2.0 to 10.0% based on the total weight of the final composition.

A second embodiment of the invention relates to the cosmetic formulation containing the composition containing AHAs and a PHA.

A third embodiment of the present invention relates to the use of said cosmetic composition in dermatological antiaging treatments with the aim of promoting cell renewal and preventing the occurrence of adverse effects related to skin irritation.

More details on these characteristics of the invention will be provided below:

### BRIEF DESCRIPTIO OF THE FIGURES

FIG. 1 represents the concentration-cell viability curve of the AHAs cosmetic formulation.

FIG. 2 shows the effect of the AHAs cosmetic formulation on histamine production in culture of human keratinocytes incubated concomitantly with interleukin-lalpha (IL-1α).

FIG. 3 shows the mean values obtained for the ITA° (Individual Typology Angle) at each time of evaluation, for the AHAs cosmetic formulation and for the control.

FIG. 4 shows the mean values obtained for the CRI (Cell Renewal Index) after 7, 14 and 28 days for the AHAs cosmetic formulation and for the control.

### DETAILED DESCRIPTION OF THE INVENTION

This is a description of a cosmetic composition and its use as a cell renewal promoting agent. Said composition comprises the combination of one or more PHAs with one or more AHAs in order to reduce the symptoms of dermatological disorders resulting from the use of AHAs. Additionally, said composition may comprise the use of various cosmetically-active ingredients with known soothing action.

Surprisingly, the inventors of the present invention have noted that said composition promotes cell renewal of the skin stratum corneum and performs a prevention/inhibition action of histamine synthesis at a high concentration of alpha-hydroxy acids

The formulation of the composition is carried out by combining one or more polyhydroxy acids with one or more alpha hydroxy compounds (AHAs) which act as keratolytic agents.

Examples of the alpha-hydroxy compounds which may be employed are benzylic acid, citric acid, glycolic acid, lactic acid, malic acid, mandelic acid, tartaric acid, or a mixture thereof.

Examples of polyhydroxy acid compounds which may be employed are gluconic acid, lactobionic acid and gluconolactone.

Preferably, the composition formulation is performed by combining at least one polyhydroxy acid with at least four alpha hydroxy compounds (AHAs)

More specifically, PHA and the lactobionic acid and the alpha-hydroxy compounds (AHAs) are employed as a mixture of glycolic acid, lactic acid, mandelic acid and malic acid.

In addition to the alpha-hydroxy compounds, other active ingredients such as glycyrrhizic acid or a cosmetically-acceptable derivative thereof, alpha-bisabolol and rhamnosoft (biosaccharide gum) may be additionally used in the formulation. These assets possess soothing action known from the prior art.

AHAs are carboxylic acids belonging to the family of organic acids. These compounds have a terminal carboxyl group, one or two hydroxyl groups attached to the first carbon (alpha position) and a carbonic chain of variable length. They can be obtained from natural sources, such as fruits, sugar cane and honey, or can be synthesized in the laboratory.

Glycolic Acid is the most commonly used AHA in cosmetics. It is derived from sugarcane and has a great ability to penetrate the epidermis, reducing the thickness of the hyperkeratinous corneal layer and promoting the reduction of cohesion between the corneocytes and their layers. In addition, it has exfoliating effect on the skin, providing whitening and stimulating the synthesis of collagen in the dermis. In this way, glycolic acid acts in the reversion and prevention of skin aging, in the improvement of age spots, stretch marks, acne scars, in addition to the therapeutic application of warts and peels.

Lactic acid can be obtained by bacterial fermentation of lactose, which is milk sugar. Furthermore, it is also produced by the human body and is part of the skin's natural moisturizing system, favoring the elasticity of the fibers, providing cell renewal and acting as a rejuvenator and whitening agent.

Mandelic acid is derived from the hydrolysis of the bitter almond extract. It is the AHA with higher molecular weight which makes its dermal absorption slower and homogeneous. This more homogeneous action results in a less irritative effect than the other AHAs. In addition, mandelic acid helps reverse the collagen degeneration caused by solar radiation, and is widely used in cosmetics aimed at skin rejuvenation.

Malic acid is one of the natural sources of alpha-hydroxy acids. It is found in nature in fruits like apple and pear. In the pharmaceutical industry, it is used in the sanitation and regeneration of wounds and burns. Furthermore, it can also increase the production of collagen and fight skin aging caused by solar radiation.

Alpha-hydroxy compounds have low molecular weight molecules and therefore are characterized by rapid penetration into the skin, which can cause stinging and cutaneous burning.

Unlike alpha-hydroxy acids (AHAs), polyhydroxyacids (PHAs) have two or more hydroxyl groups, not necessarily in the alpha position, forming an aliphatic or alicyclic molecular structure (Yu & Van Scott, 1996). Because they have larger molecular structures, PHAs penetrate the skin more smoothly and gradually than AHAs, bypassing the adverse effects related to skin irritation.

Although this action is expected, it is entirely unexpected that the addition of a PHA compound in a cosmetic formulation containing AHAs may exhibit inhibition of the skin irritation effects normally exhibited by AHA compounds, especially when they are in high concentrations.

According to the present invention, the terms “cosmetically-active ingredient”, “active ingredient” and “active” are used interchangeably and refer to the compounds in a composition which promote a desired cosmetic effect.

According to the present invention, the term “cosmetically-acceptable” refers to compounds which are commonly used in the cosmetic art together with active ingredients. In particular, “cosmetically-acceptable” refers to compounds which confer, without limitation, shape, aroma, stability and coloration to the final composition, in a safe and tolerable way for a user of the final product. In some embodiments, a “cosmetically-acceptable” component may facilitate the absorption of one or more active ingredients under application.

Cosmetically-acceptable excipients include, without limitation, pH adjusting agents, conditioners, preservatives, thickeners, emollients, emulsifiers, absorbents, binders, fragrances, film-forming agents, solvents, humectants, antioxidants, viscosity controlling agents, surfactants, sequestrants and vehicles.

Examples of pH adjusting agents include, without limitation, aminomethylpropanol (AMP), sodium bicarbonate, ammonium carbonate, potassium hydroxide, sodium hydroxide, triethanolamine, sodium phosphate monobasic and sodium phosphate dibasic.

Examples of conditioners include, without limitation, glycyrrhizic acid, bisabolol, caprylyl methacone and biosaccharide gum-2.

Examples of preservatives include, without limitation, phenoxyethanol, imidazolidinyl urea, ethylhexylglycerine, methylisothiazolinone, methylchloroisothiazolinone, sodium benzoate, benzoic acid, benzyl alcohol, butylparaben, cetylpyridinium chloride, benzalkonium chloride, benzethonium chloride, ethylparaben, methylparaben, propylparaben, and mixtures thereof.

Examples of thickeners include, without limitation, waxes, such as beeswax, carnauba, candelilla and lanolin wax, polysaccharides, among which starch, gums such as gum arabic, guar gum, xanthan gum, tragacanth, agar-agar, carrageenans and alginates, cellulose and its derivatives, such as microcrystalline cellulose, cellulose acetate, carboxymethylcellulose and hydroxyethylcellulose, glyceryl stearate, polyethylene glycol, polyvinylpyrrolidone, polyvinyl alcohol, carbopol, polyacrylic acid, silanes and derivatives, alkyl polyacrylates, alkyl polymethacrylates and mixtures thereof.

Examples of emollient agents include, without limitation, isononyl isonone, di-isopropyl sebacate, stearic acid and animal fats such as lanolin.

Examples of emulsifiers include, without limitation, ethoxylated fatty esters, such as PEG-20 triperrenyl, PEG-100 stearate, fatty acid mono- and diesters, such as glyceryl stearate, oleic acid and derivatives, fatty alcohols such as cetostearyl alcohol, cetyl alcohol , as well as anionic surfactants, such as sodium dodecylsulfate and sodium lauryl ether sulfate.

Examples of absorbents include, without limitation, silica and starch aluminum octenyl succinate.

Examples of binders include, without limitation, sodium chloride, guar gum, hydroxyethylcellulose, and PEG-90M.

Examples of fragrances include, without limitation, natural, synthetic fragrances and mixtures thereof.

Examples of film forming agents include, without limitation, polysilicone-11; synthetic or natural cationic polymers such as quaternized guar gum, polyquaterniums and chitosan; acrylates and polymers, cellulose and derivatives.

Examples of solvents include, without limitation, alcohols, such as ethoxydiglycol, propanediol, phenylpropanol, butylene glycol and pentylene glycol.

Examples of humectants include, without limitation, ethoxydiglycol, glycerin, lactose, urea, and hydrolyzed hyaluronic acid.

Examples of antioxidants include, without limitation, lactobionic acid, benzotriazolyl dodecyl p-cresol and octadecyl di-t-butyl-4-hydroxyhydrocinnamate.

Examples of viscosity controlling agents include, without limitation, alcohols, such as propylene glycol and butylene glycol; natural polymers such as cellulose and derivatives, carrageenan and derivatives; or synthetic polymers, such as polymers and acrylic crospolymers.

Examples of surfactants include, without limitation, nonionic ones, such as lauret-12, lauret-23; ionic acids such as sodium lauryl sulfate, sodium lauryl ether sulfate, and amphoterics such as betaines and hydroxysultains.

Examples of sequestrants include, without limitation, EDTA, disodium EDTA, tetrasodium EDTA, and mixtures thereof.

Examples of carriers include, without limitation, water, alcohols such as ethanol, phenylpropanol and propanediol and mixtures thereof.

The cosmetic composition may be provided in a number of forms, including, without limitation, aerosol, cream, gel, lotion and serum. A person skilled in the art will recognize that the cosmetic form will be defined by the choice of the cosmetically-acceptable excipients and that the absorption/action of the different active ingredients may vary according to the selected cosmetic form.

The concentrations for each active ingredient of the cosmetic formulation of the present invention are described below.

Glycolic acid is present in a concentration range of 2.0-10.0% based on the total weight of the final composition.

Lactic acid is present in a concentration range of 0.5-3.0% based on the total weight of the final composition.

Malic acid is present in a shrinkage range of 2.0-4.0% based on the total weight of the final composition.

Mandelic acid is present in a concentration range of 2.0-10.0% based on the total weight of the final composition.

The lactobionic acid is present in a concentration range of 2.0-10.0% based on the total weight of the final composition.

### EXAMPLES

The following examples illustrate the preferred but not limiting embodiments of the present invention. It is not to be understood, however, that they limit the scope of protection of the invention, which is solely defined by the claims accompanying this disclosure.

### Example 1

### Preparation of the Formulation of the Present Invention

1—Add water and solubilize the EDTA in the main reactor (aqueous phase).

2—Solubilize lactobionic acid, hydrolyzed hyaluronic acid and malic acid

3—Add 70% glycolic acid and lactic acid.

4—Add propanediol. Heat to 35° C., add mandelic acid.

5—Adjust the pH to 3.8 with AMP. Add sodium chloride.

6—Add polyacrylate-6 crospolymer, wait ca 40′ under low stirring. Add xanthan gum, wait ca 30′.

7—Heat to 75-80° C.

8—In an auxiliary reactor, add the oil phase: triberrenil PEG-20, stearate PEG-100, glyceryl stearate, laureth-23, benzotriazolyl dodecyl p-cresol, octadecyl di-t-butyl-4-hydroxyhydrocinnamate, isononyl isononanoate, sebacate diisopropyl, caprylyl methicone, alpha bisabolol, ethoxydiglycol.
Heat to 75-80° C.

9—Pour oily phase into aqueous Maintain temperature for ca 5′. Start cooling.

10—Add phenoxyethanol and methylisothiazolinone below 35° C.,

11—Add glycyrrhizic acid and alcohol.

12—Disperse the HDl/trimethylol hexylactone and silica, aluminum starch octenyl succinate crospolymer in 2-biosaccharide gum, polysilicon-11, water, laureth-12, phenoxyethanol, ethylhexylglycerin and PEG-90M and add the mixture to the main reactor under agitation.

13—Add the fragrance, shake for ca 5 minutes.

15—Verify final pH. It must be from 3.5 to 4.0. If necessary, adjust pH to 3.8 with AMP.

The compounds used in the formulation of the cosmetic composition of the present invention, their wt./wt. percentages and their respective functions are set forth in Table 1.

TABLE 1

| Compound | % wt/wt | Function |
|---|---|---|
| Water | 53.95 | Carrier |
| Propanediol | 5.00 | Solvent |
| Aminomethylpropanol | 4.75 | PH adjustment |
| Triberrenil PEG-20 | 4.50 | Emulsifier |
| Alcohol | 3.00 | Solvent |
| Glycolic Acid | 2.80 | Keratolytic agent |
| Isononyl Isononanoate | 2.50 | Emollient agent |
| Lactobionic Acid | 2.00 | Antioxidant/Humectant |
| | | agent |
| Malic Acid | 2.00 | Keratolytic agent |
| Mandelic Acid | 2.00 | Keratolytic agent |
| Caprylyl Methicone | 2.00 | Skin Conditioner |
| HDl/Trimethylol | 1.96 | Deagglutinant agent |
| Hexylactone Crospolymer | | |
| Diisopropyl Sebacate | 1.80 | Emollient agent |
| Lactic Acid | 1.70 | Keratolytic agent |
| PEG-100 Stearate | 1.20 | Emulsifier |
| Glycyrrhizic Acid | 1.00 | Skin Conditioner |
| Bisabolol | 1.00 | Skin Soothing Agent |
| Polyacrylate-6 | 1.00 | Viscosity Controller |
| Crospolymer | | |
| Glyceryl Stearate | 0.88 | Emulsifier |
| Starch Aluminum | 0.80 | Absorbent Agent |
| Octenylsuccinate | | |
| Polysilicone-11 | 0.73 | Film Forming Agent |
| Hydrolyzed Hyaluronic | 0.50 | Humectant Agent |
| Acid | | |
| Sodium Chloride | 0.50 | Agglomerating Agent |
| Ethoxydiglycol | 0.50 | Solvent |
| Xanthan Gum | 0.50 | Thickening Agent |
| Phenoxyethanol | 0.50 | Preservatives |
| Perfume | 0.28 | Fragrance |
| Lauret-23 | 0.13 | Surfactant |
| Benzotriazolyl Dodecyl p- | 0.10 | Antioxidant |
| Cresol | | |
| Octadecyl di-t-butyl-4- | 0.10 | Antioxidant |
| Hydroxyhydrocinnamate | | |
| Disodium EDTA | 0.10 | Sequestrant |
| Biosaccharide Gum-2 | 0.10 | Skin Conditioner |
| Propanediol | 0.06 | Carrier |
| Silica | 0.04 | Absorbent Agent |
| Lauret-12 | 0.01 | Surfactant |
| PEG-90M | 0.01 | Binder |
| Methylisothiazolinone | 0.008315 | Preservatives |
| Imidazolidinyl Urea | 0.0015 | Preservatives |
| Ethylhexylglycerin | 0.0012 | Preservatives |
| | 100.00 | |

### Example 2

### Cell Viability Test

The cell viability test was performed with the objective of determining the non-cytotoxic concentrations of the cosmetic AHA formulation of the present invention.

Cell viability was determined by a colorimetric method using the XTT dye, which is converted to water-soluble orange formazan by the enzyme mitochondrial succinate dehydrogenase in viable cells (Xenometrix AG, Switzerland). Fibroblasts were seeded at the density of 1×104 cells per well and incubated with the cosmetic AHA formulation at 8 concentrations using a decimal geometric dilution. After 48 hours of incubation, the cosmetic formulation of the present invention was removed, and the culture medium was replaced. XTT was then added to the culture and the plate incubated for an additional 3 hours. The absorbance (OD) of each well was determined at 480 nm in Multiskan GO monochromator (Thermo Scientific, Finland). The percentage of cell viability was calculated according to the equation:

% Viability=(DO_{FC}/DO_{CN})×100,

where DOFC=Optical density of the cosmetic formulation of the present invention and DOCN=optical density of the negative control.

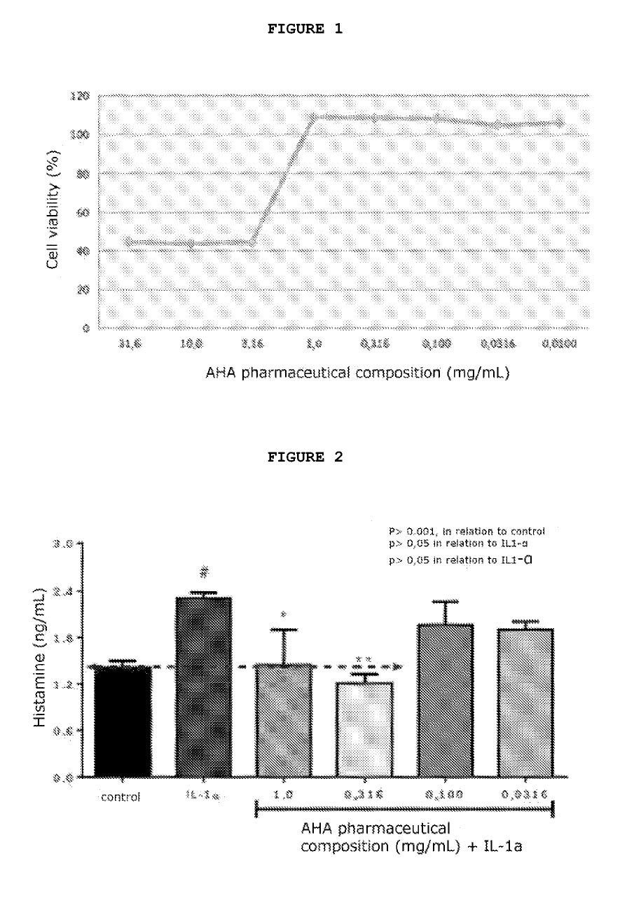

FIG. 1 represents the concentration-cell viability curve of the AHA cosmetic formulation of the present invention.

The results shown in FIG. 1 demonstrate that the cosmetic AHA formulation of the present invention showed non-cytotoxic concentrations from the 1.0 mg/mL dilution.

### Example 3

### Quantification of Histamine

Keratinocyte cultures were incubated with 4 non-cytotoxic concentrations of the cosmetic formulation of the present invention determined by the cytotoxicity assay. The concentrations assessed in this study were 1.0; 0.316; 0.100 and 0.0316 mg/mL. Inflammatory stress was mimicked by the addition of IL-1α—10 ng/ml to keratinocyte cultures concomitantly with the treatment with the cosmetic formulation of the present invention. Cells were maintained in culture for 48 hours. After this time, the cell culture supernatant was collected for quantification of the proposed mediator.

The histamine concentration was measured by competitive ELISA using a commercially available kit (Oxford Biomedical Research, Oxford, Mich., United States of America). The absorbance reading was performed in Multiskan GO monochromator (Thermo Fischer Scientific, Vantaa, Finland).

In the statistical evaluation, we used the ANOVA test that allowed to measure the variation of the results, comparing the data between the groups, evidencing or not the differences between them. The non-parametric Tukey test was applied, which reinforced and made even more precise the presented result. A 5% significance level was used.

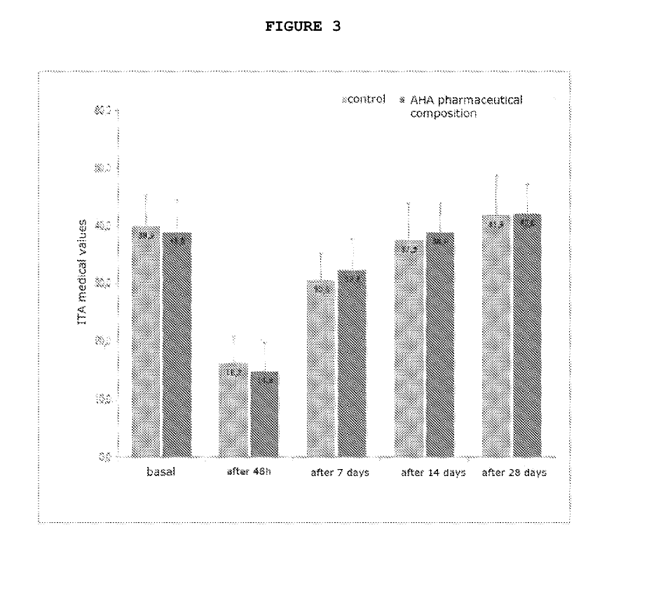

FIG. 2 shows the effect of the AHAs cosmetic formulation of the present invention on histamine production in culture of human keratinocytes incubated concomitantly with interleukin-lalpha (IL-1α). Data represent the mean±standard deviation of 3 replicates (Anova, Tukey).

IL-1α promotes a statistically significant increase (P <0.001) in histamine synthesis, which is prevented by the concomitant addition of the cosmetic formulation of the present invention in cell cultures. At concentrations of 1.0 and 0.316 mg/mL, the AHAs cosmetic formulation prevents up to 100% increase in IL-1α-induced histamine synthesis, maintaining levels similar to those in the control group, as can be observed in the dotted line.

The results indicate that the use of the cosmetic formulation prevents the synthesis of histamine in the cellular microenvironment after inflammatory stress induced by IL-1α, thus avoiding the appearance of reactions of irritation, pruritus and erythema.

### Example 4

### Cellular Renewal

The ability to measure biological aging based on structural and functional changes in the skin, without using invasive methods, suggests a breakthrough in science in this area.

Some noninvasive methods have been developed to determine the renewal time of the stratum corneum. These methods use compounds that react with structures of the stratum corneum, generating by-products that, depending on their coloration fluorescence emission, can be objectively measured by in vivo colorimetry or fluorescence spectroscopy.

The efficacy of the cosmetic formulation was assessed by varying the color of the skin as measured by a colorimetric technique.

Dihydroxyacetone (DHA) is a 3-carbon reducing sugar that reacts with the free amino groups of keratin generating dark-colored by-products called melanoidins, by non-enzymatic darkening, Maillard reaction (Levy, 2000). DHA penetrates the stratum corneum reaching the upper layers of the granular layer, forms the melanoidines and their elimination is limited to natural skin peeling or physical removal of the corneocytes (Forest et al, 2003).

The tristimulus colorimeter is an instrument that provides measurements correlated to the perception of the human eye through tristimulus values (XYZ, L, a, b, etc).

The study was based on the hypothesis that application of the cosmetic formulation of the present invention could confer increased cell renewal of the stratum corneum of the skin.

Stimulation of cell turnover was measured indirectly by colorimetric evaluation of an area treated by the cosmetic formulation of the present invention as compared to the control area (without any treatment).

The colorimetric assessment of the skin was performed at the beginning of the study (prior to the application of the cosmetic formulation of the present invention and skin darkening), after 48 hours of the application of 10% dihydroxyacetone (DHA) (for color stabilization) and after 7, 14, and 28 days of home use of the cosmetic formulation of the present invention.

The study consists of DHA application in an area of 2.5×4.0 cm, demarcated in one of the forearms of several volunteers. The application of DHA promotes the darkening of the area of application in order to allow the analysis of the cellular renewal of the stratum corneum of the skin. The application did not occur in the other forearm, referred to as the control area.

Prior to the DHA application, the skin was cleaned with a 10% hydroalcoholic solution to remove any dirt residues and to remove loose corneocytes. Then, with the aid of a micropipette, 30 μl of an emulsion containing 10% DHA were applied. The emulsion was spread homogeneously within the limits of the demarcated area in the volunteers' forearms.

The emulsion application procedure was repeated two more times, with an interval of 1 hour between the applications. After the applications, the research participants returned home, and were instructed not to expose themselves to the sun, apply any products or wash their forearms. After 48 hours of the last application of DHA, the participants returned to the laboratory to make measurements of skin color.

The measurements were performed using the Konica Minolta CR-400 colorimeter. Skin pigmentation was measured using the L* and b* parameters measured by the colorimeter, determining the ITA° (Individual Typology Angle) at the beginning of the study (before the application of DHA and the cosmetic formulation of the present invention), after 48 hours of the last application of DHA, and after 7, 14 and 28 days of use of the cosmetic formulation of the present invention. ITA° values can be calculated by the following formula:

ITA°=(Tangent Arch ((L−50)/b)]*180/π

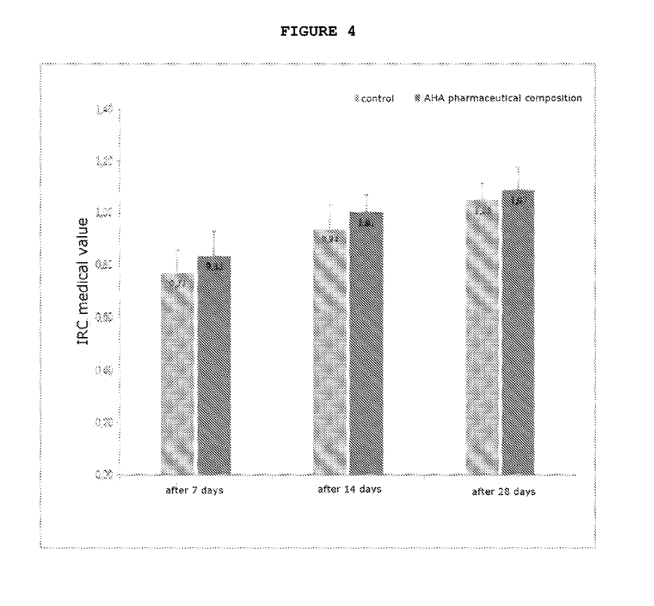

FIG. 3 shows the mean values obtained for the ITA° at each time of evaluation, for the AHAs cosmetic formulation and for the control.

The reduction of ITA° values indicates skin darkening and, therefore, a decrease in ITA° values is expected after 48 hours of DHA application, and gradual return to baseline during the course of the study.

To evaluate the stimulation of the cellular renewal provided by the cosmetic formulation as a function of the control, the Cell Renewal Index (CRI) was calculated, according to the following equation:

CRI=ITA°ₓₜᵢ/ITA°ₓₜ₀,

where x=cosmetic formulation or control, ti=measurement time and to=start of the study (prior to application of the DHA and the cosmetic formulation of the present invention).

FIG. 4 shows the mean values obtained for the CRI after 7, 14 and 28 days for the AHAs cosmetic formulation and for the control.

Loss of skin color indicates cell renewal. Thus, the efficacy of the cosmetic formulation is proven when its application to the skin promotes the return of the ITA° value to its basal value more quickly than in the control forearm. Based on the results shown in FIG. 3 and FIG. 4, it is realized that the application of the cosmetic formulation of the present invention accelerates the process of cell renewal and exfoliation, providing the cellular renewal of the skin just after 14 days of use.

Considering that the natural process of cell renewal of the stratum corneum is approximately 28 days, one can state that the cosmetic formulation of the present invention has a twice as fast skin cell renewal rate.

From the foregoing description and examples, it is possible to observe an unexpected improvement in the prevention/inhibition of histamine synthesis, while maintaining the therapeutic efficacy of the cosmetic formulation disclosed herein with respect to the prior art.

While certain embodiments have been specifically described, they have been disclosed only by way of example, and there is no intention to limit the scope of the invention. The claims accompanying this disclosure and its equivalents are considered to encompass such embodiments.

Finally, modifications of the present invention, apparent to one skilled in the art, such as addition or removal of non-fundamental elements to its embodiment, may be performed without departing from the scope and spirit of the invention.

1. A cosmetic composition characterized by comprising one or more alpha-hydroxy acid(AHA) compounds and by further comprising one or more polyhydroxy acid (PHAs) compounds.

2. A cosmetic composition according to claim 1, characterized in that it comprises at least four alpha hydroxy compounds (AHAs) and additionally comprising at least one polyhydroxy acid compound (PHA).

3. The cosmetic composition according to claim 1, characterized in that the AHA compounds are selected from benzylic acid, citric acid, glycolic acid, lactic acid, malic acid, mandelic acid, tartaric acid, or a mixture thereof.

4. The cosmetic composition according to claim 1, characterized in that the PHAs are selected from gluconic acid, lactobionic acid, gluconolactone, or a mixture thereof.

5. The cosmetic composition according to claim 1 characterized in that the AHA compound or the AHA mixture is present in an amount of 0.1 to 10.0% by weight of the total composition.

6. The cosmetic composition according to claim 1, characterized in that the AHA mixture comprises glycolic acid, lactic acid, malic acid and mandelic acid.

7. The cosmetic composition according to claim 6, characterized in that the glycolic acid is present in a concentration range of 2.0 to 10.0% by weight of the total composition.

8. The cosmetic composition according to claim 6, characterized in that the lactic acid is present in a concentration range of 0.5 to 3.0% by weight of the total composition.

9. The cosmetic composition according to claim 6, characterized in that the mandelic acid is present in a concentration range of 2.0 to 10.0% by weight of the total composition.

10. The cosmetic composition according to claim 6, characterized in that the malic acid is present in a concentration range of 2.0 to 4.0% by weight of the total composition.

11. The cosmetic composition according to claim 1, characterized in that the PHA compound or the PHA mixture is present in an amount of 0.1 to 10.0% by weight of the total composition.

12. The cosmetic composition according to claim 1, characterized in that the compound PHA is lactobionic acid.

13. The cosmetic composition according to claim 12, characterized in that the lactobionic acid is present in a concentration range of 2.0 to 10.0% by weight of the total composition.

14. The cosmetic composition according to claim 1, characterized by further comprising one or more cosmetically-active ingredients selected from glycyrrhizic acid or a cosmetically-acceptable derivative thereof, alpha-bisabolol, biosaccharide gum, or a mixture thereof.

15. A cosmetic formulation characterized by comprising a composition according to claim 1 and by being in the form aerosol, cream, gel, lotion or serum.

16. The cosmetic formulation according to claim 15, characterized by comprising the composition in a concentration ranging from 0.316 to 1000 mg/ml.

17. Use of the cosmetic composition of claim 1 characterized by playing the role of prevention/inhibition of histamine synthesis.

18. Use according to claim 17, characterized in that it prevents irritation, pruritus and erythema reactions.
